(19) United States
(12) Patent Application Publication
(10) Pub. No.: US 2021/0332037 A1
(43) Pub. Date: Oct. 28, 2021

(54) Scopolamine Production

(71) Applicant: The University of Johannesburg, Johannesburg (ZA)

(72) Inventors: Pangaman Jiyane, Randburg (ZA); Derek Tantoh Ndinteh, Windsor (ZA)

(73) Assignee: The University of Johannesburg, Johannesburg (ZA)

(21) Appl. No.: 17/291,688

(22) PCT Filed: Nov. 6, 2019
(86) PCT No.: PCT/ZA2019/050069

§ 371 Date: May 6, 2021

(30) Foreign Application Priority Data
Nov. 7, 2018 (ZA) 2018/07451

(51) Int. Cl. C07D 451/10

(52) U.S. Cl. CPC C07D 451/10

(57) ABSTRACT

The invention provides an improved method for the production of Scopolamine by extraction.

### TECHNICAL FIELD OF THE INVENTION

This invention relates to the production of Scopolamine. More particularly it relates to the production of Scopolamine by means of extraction from plant material.

### BACKGROUND TO THE INVENTION

Scopolamine, also known as Hyoscine, is a well-known drug used inter alia for motion sickness and nausea. It is also a precursor to a number of other drugs. Scopolamine is expensive to synthesize and it is therefore economically viable to be extracted from plants of the Solanaceae family.

Previously, the applicant invented an extraction and purification method of scopolamine in flos daturae. The method included the following steps: firstly obtaining extract liquid of flos daturae; filtering the extract liquid by means of a ceramic membrane, concentrating by means of a nanofiltration membrane, recovering ethanol, and obtaining a mixture; dissolving the mixture by using diluted hydrochloric acid, carrying out suction filtering, and obtaining filtrate; extracting the filtrate, and reserving the aqueous solution; adjusting the pH value by using a water phase, re-extracting four times, and collecting a chloroformic solution; drying the chloroformic solution, recovering chloroform, and obtaining total alkaloids; and separating the total alkaloids by using alkaline aluminum oxide column chromatography, recovering chloroform, and obtaining scopolamine.

The applicant wishes to improve the yield and the scalability of the above method.

It is an object of the invention to provide a simplified method to produce Scopolamine which is well suited for scale up to process 45 000 kg or more of plant material per year.

### GENERAL DESCRIPTION OF THE INVENTION

According to the invention there is provided a method for the production of Scopolamine, which method includes the steps of:

grinding plant material which contains Scopolamine;

adding the grinded plant material to a Sulfuric acid solution, preferably 1%, and allowing to stand in the solution for 1 to 4, preferably 2 to 3 days;

filtration separation of the acid solution from the plant material;

repeating the previous steps with the residue plant material and combining the acidic filtrates;

neutralising with sodium bicarbonate to a pH of between 7.5 and 9.5, preferably between 8 and 9;

addition, preferably immediate, of dichloromethane for the extraction of the alkaloids in the neutralised solution to the organic layer and allowing to stand for 1 to 4 days, preferably 2 to 3 days;

separation of the bottom organic layer from the upper aqueous layer;

evaporation of the dichloromethane solvent to obtain the coloured scopolamine rich extract;

adding the scopolamine rich extract to dichloromethane solvent and mixing with a NaOH, preferably 1%, solution and separating the layers;

evaporation of the dichloromethane solvent to obtain the less coloured scopolamine rich extract;

optionally, adding the less coloured scopolamine rich extract to dichloromethane solvent and mixing with a sodium bicarbonate, preferably 1%, solution and separating the layers; and

evaporation of the dichloromethane solvent to obtain the final scopolamine rich extract.

The second last optional step improves the purity of the final product.

The plant material may preferably be the seeds of Flos Daturae. It is to be appreciated that the seeds have less chlorophyll, which follows the extraction process to some extent.

The extraction method described is significantly less complex than known methods, does not need special equipment like columns and separation membranes, uses only one low cost solvent and produces a high yield with high purity. The method can also be economically scaled up as required to process 45 tons or more of plant material per year. The extraction method is also tuned to focus on scopolamine from a mixture of alkaloids contained in Flos Daturae.

### DETAILED DESCRIPTION OF THE INVENTION

The invention is now described by way of example.

500 Grams of Flos Daturae seeds were grinded and added to 1 500 ml of a 1% Sulfuric acid solution and allowed to stand in the solution for 2 days. The mixture was filtered and 700 ml of the acid solution was recovered. This step can preferably be repeated to increase the yield. The filtrate was neutralised with sodium bicarbonate to a pH of 9. A 1000 ml of dichloromethane was mixed with the filtrate and allowed to stand for 2 days. The bottom organic layer was separated from the upper aqueous layer and the dichloromethane solvent was evaporated using a roto evaporator to obtain the coloured scopolamine rich extract. Once off, adding the scopolamine rich extract to 1000 ml dichloromethane solvent and mixing with a 1% NaOH 1000 ml, solution and separating the layers. The dichloromethane solvent was again removed in a roto evaporator to obtain the less coloured scopolamine rich extract. The less coloured scopolamine rich extract was added to 1000 ml dichloromethane solvent and mixed with a 1000 ml 1% sodium bicarbonate solution and the layers were separated. This step is also once off. The dichloromethane solvent was evaporated to obtain 12 grams of the final scopolamine rich extract, which contains 70% scopolamine in a pharmaceutically acceptable mixture.

The use of sulphuric add in the first step removes fat soluble impurities. The long extraction time of 2 days improves the yield significantly and it appears that the conversion from organic acid to salt is relatively slow. The selective neutralisation to pH of 8 to 9 avoided neutralising other compounds and their subsequent extraction to yield a purer product. Again, longer than normal extraction time improved the yield and the further steps improved the purity compared to known extraction methods.

It shall be understood that the example is provided for illustrating the invention further and to assist a person skilled in the art with understanding the invention and is not meant to be construed as unduly limiting the reasonable scope of the invention.

1. A method for the production of Scopolamine, which method includes the steps of:
grinding plant material which contains Scopolamine;
adding the grinded plant material to a Sulfuric acid solution and allowing to stand in the solution for 1 to 4 days,
filtration separation of the acid solution from the plant material;
repeating the previous steps with the residue plant material and combining the acidic filtrates;
neutralising with a alkaline solution to a pH of between 7.5 and 9.5;
addition of dichloromethane for the extraction of the alkaloids in the neutralised solution to the organic layer and allowing to stand for 1 to 4 days;
separation of the bottom organic layer from the upper aqueous layer;
evaporation of the dichloromethane solvent to obtain the coloured scopolamine rich extract;
adding the scopolamine rich extract to dichloromethane solvent and mixing with a NaOH solution and separating the layers; and
evaporation of the dichloromethane solvent to obtain the less coloured scopolamine rich extract.

2. The method for the production of Scopolamine as claimed in claim 1, wherein the Sulfuric acid solution is 1%.

3. The method for the production of Scopolamine as claimed in claim 1, wherein the Sulfuric acid solution is allowed to stand for 2 to 3 days.

4. The method for the production of Scopolamine as claimed in claim 2, wherein the Sulfuric acid solution is allowed to stand for 2 to 3 days.

5. The method for the production of Scopolamine as claimed in claim 1, wherein the alkaline solution is a sodium bicarbonate solution at a pH of between 8 and 9.

6. The method for the production of Scopolamine as claimed in claim 2, wherein the alkaline solution is a sodium bicarbonate solution at a pH of between 8 and 9.

7. The method for the production of Scopolamine as claimed in claim 3, wherein the alkaline solution is a sodium bicarbonate solution at a pH of between 8 and 9.

8. The method for the production of Scopolamine as claimed in claim 1, wherein the Dichloromethane is added immediately.

9. The method for the production of Scopolamine as claimed in claim 2, wherein the Dichloromethane is added immediately.

10. The method for the production of Scopolamine as claimed in claim 3, wherein the Dichloromethane is added immediately.

11. The method for the production of Scopolamine as claimed in claim 5, wherein the Dichloromethane is added immediately.

12. The method for the production of Scopolamine as claimed in claim 1, wherein the organic layer is allowed to stand for 2 to 3 days.

13. The method for the production of Scopolamine as claimed in claim 2, wherein the organic layer is allowed to stand for 2 to 3 days.

14. The method for the production of Scopolamine as claimed in claim 3, wherein the organic layer is allowed to stand for 2 to 3 days.

15. The method for the production of Scopolamine as claimed in claim 5, wherein the organic layer is allowed to stand for 2 to 3 days.

16. The method for the production of Scopolamine as claimed in claim 8, wherein the organic layer is allowed to stand for 2 to 3 days.

17. The method for the production of Scopolamine as claimed in claim 1, wherein the NaOH solution is 1%.

18. The method for the production of Scopolamine as claimed in claim 2, wherein the NaOH solution is 1%.

19. The method for the production of Scopolamine as claimed in claim 3, wherein the NaOH solution is 1%.

20. The method for the production of Scopolamine as claimed in claim 5, wherein the NaOH solution is 1%.

21. The method for the production of Scopolamine as claimed in claim 8, wherein the NaOH solution is 1%.

22. The method for the production of Scopolamine as claimed in claim 12, wherein the NaOH solution is 1%.

23. The method for the production of Scopolamine as claimed in claim 1, wherein the less coloured scopolamine rich extract is added to dichloromethane solvent and mixed with a 1% sodium bicarbonate solution and the layers separated and the dichloromethane solvent evaporated to obtain the final scopolamine rich extract.

24. The method for the production of Scopolamine as claimed in claim 2, wherein the less coloured scopolamine rich extract is added to dichloromethane solvent and mixed with a 1% sodium bicarbonate solution and the layers separated and the dichloromethane solvent evaporated to obtain the final scopolamine rich extract.

25. The method for the production of Scopolamine as claimed in claim 3, wherein the less coloured scopolamine rich extract is added to dichloromethane solvent and mixed with a 1% sodium bicarbonate solution and the layers separated and the dichloromethane solvent evaporated to obtain the final scopolamine rich extract.

26. The method for the production of Scopolamine as claimed in claim 5, wherein the less coloured scopolamine rich extract is added to dichloromethane solvent and mixed with a 1% sodium bicarbonate solution and the layers separated and the dichloromethane solvent evaporated to obtain the final scopolamine rich extract.

27. The method for the production of Scopolamine as claimed in claim 8, wherein the less coloured scopolamine rich extract is added to dichloromethane solvent and mixed with a 1% sodium bicarbonate solution and the layers separated and the dichloromethane solvent evaporated to obtain the final scopolamine rich extract.

28. The method for the production of Scopolamine as claimed in claim 12, wherein the less coloured scopolamine rich extract is added to dichloromethane solvent and mixed with a 1% sodium bicarbonate solution and the layers separated and the dichloromethane solvent evaporated to obtain the final scopolamine rich extract.

29. The method for the production of Scopolamine as claimed in claim 17, wherein the less coloured scopolamine rich extract is added to dichloromethane solvent and mixed with a 1% sodium bicarbonate solution and the layers separated and the dichloromethane solvent evaporated to obtain the final scopolamine rich extract.
